(19) United States
(12) Patent Application Publication
(10) Pub. No.: US 2023/0142106 A1
(43) Pub. Date: May 11, 2023

(54) TRUSTLESS MONITORING AND VALIDATION

(71) Applicants: Eric DOWNES, West Palm Beach, FL (US); Asra Ali, Austin, TX (US); Brian Neltner, Sommerville, MA (US)

(72) Inventors: Eric DOWNES, West Palm Beach, FL (US); Asra Ali, Austin, TX (US); Brian Neltner, Sommerville, MA (US)

(21) Appl. No.: 17/974,471

(22) Filed: Oct. 26, 2022

Related U.S. Application Data
(60) Provisional application No. 63/272,113, filed on Oct. 26, 2021.

(51) Int. Cl. G01N 33/00; H04L 9/00; G01N 33/18

(52) U.S. Cl. CPC G01N 33/004; H04L 9/008; G01N 33/0073; H04L 9/50; G01N 33/0047; G01N 33/18

(57) ABSTRACT

Systems and methods to trustlessly provide resource consumption and/or pollution emission readings at cooperating industrial, commercial, or consumer locations, including mechanisms for trustless blockchain-based verification by third parties by employing a measure-perturb-measure sensor validation cycle, and to safe guard both the privacy and value of collected data during an adjustable pre-determined lifecycle.

### CROSS-REFERENCE TO RELATED APPLICATIONS

This application claims priority to U.S. Prov. Appl. No. 63/272,113, filed Oct. 26, 2021, which is hereby incorporated by reference in its entirety.

### BACKGROUND OF THE INVENTION

Many solutions to complex system problems require cooperation among a large number of parties (countries, companies, individuals), and require trust that the other party is independently taking prescribed actions. Even when regulatory solutions to these problems are available, there are privacy concerns, and the solution can be very costly and subject to regulatory capture.

Self-reporting users, and even third-party observers meant to be objective, are part of a trust-based monitoring solution. It is possible to construct automated electronic systems designed to be trustless. In a trustless system the participants involved do not need to know or trust each other, or trust a third party, for the system to function, e.g., provision or support the desired amenity, such as data and transparency.

The Internet of Things (IoT) describes the network of physical objects—a.k.a. “things”—that are embedded with sensors, software, and other technologies for the purpose of connecting and exchanging data with other devices and systems over the Internet. With the advent of the IoT, low-power sensors, and low-cost stand-alone micro-processors, a wide-variety of environmental monitoring systems can collect data from the real-world and report in aggregate via internet gateways for secure data collection. In this way, activities can be monitored by objective observers.

### BRIEF SUMMARY OF THE INVENTION

Disclosed herein are systems and methods for controlled monitoring of activity at an installation site through a process of two distinct modes: continuous undisturbed measurements, and periodic perturbed measurements. These modes of operation are performed in series, in a cycle called measure, perturb, measure (MPM).

The measure, perturb, measure cycle is a novel process using network connected electronic sensors to trustlessly monitor and report conditions at a remote site, for example environmental conditions at a fixed location.

In a first embodiment of the invention, a system for monitoring and reporting a fluid concentration is provided. The system includes a homomorphic aggregator, a field unit including a fluid channel and a sensor sensing a concentration of one or more elements of the fluid flow, and a perturbation unit. The field unit and perturbation unit are in data communication with the homomorphic aggregator via a communication channel. The perturbation unit is in fluid communication with the fluid channel, and includes an actuator to alter the concentration of elements of the fluid flow through the channel. The field unit communicates a fluid data signal to the homomorphic aggregator via the communication channel that is representative of the concentration of one or more elements of the fluid flow. The perturbation unit receives a perturbation request and perturbs the fluid flow to the field unit, causing the field unit to provide a perturbed data signal to the homomorphic aggregator representative of the perturbed fluid concentration. The homomorphic aggregator records the fluid data signal and the perturbed data signal to a blockchain record.

In some embodiments, the field unit is configured to encrypt the fluid data signal prior to communicating the fluid data signal to the homomorphic aggregator. The perturbation request may be encrypted using a private key of the perturbation unit.

In some embodiments, the homomorphic aggregator receives the perturbation request and communicates the perturbation request to the perturbation unit. In other embodiments, the perturbation unit receives the perturbation request and communicates the perturbation request to the homomorphic aggregator.

The homomorphic aggregator may be configured to record the fluid data signal to a blockchain record at a first sampling rate and record the perturbed data signal to the blockchain record at a second sampling rate. The second sampling rate may be higher than the first sampling rate, or vice versa.

In other embodiments, the fluid data signal is added to the blockchain record using a first aggregation function, and the perturbed data signal is added to the blockchain using a second aggregation function. The blockchain record may include a data stamp indicating that perturbed data is being collected, and may include a time stamp.

In some embodiments, the fluid flow is a gas, and the component being measured may be carbon dioxide, methane, or another gas. In other embodiments, the fluid flow is water or another liquid, and the element of fluid flow being measured is a environment pollutant such as nitrites/nitrates, volatile organic compounds, dissolved O2 or CO2, or other chemicals of interest for water quality monitoring.

In another embodiment of the invention, a system for trustlessly monitoring a signal is provided. The system includes an electrical source and an electrical load, an electrical sensor, a controller, and a homomorphic aggregator. The sensor includes a sensor module coupled to the electrical load and to a controller; and the homomorphic aggregator is in data communication with the controller and the sensor module. The sensor module is configured to transmit data representative of a first operating condition of the electrical load to the homomorphic aggregator. The homomorphic aggregator records the first operating condition data to a blockchain. If the controller receives a perturbation request, the controller electronically modifies the electrical load presented to electrical source. The sensor module transmits perturbation data representative of a second operating condition to the homomorphic aggregator, and the homomorphic aggregator records the second operating condition data to the blockchain.

In some embodiments, the controller receives the perturbation request from the homomorphic aggregator. In other embodiments, the controller receives the perturbation request from an external source.

In some embodiments, the homomorphic aggregator adds the first operating condition data to the blockchain using a first aggregation function, adds the second operating condition data to the blockchain using a second aggregation function.

In still another embodiment of the invention, a method of monitoring and reporting a fluid concentration is provided. The method includes providing a homomorphic aggregator with a processor and an encrypted communication channel, a field unit, and a perturbation unit. The field unit and the perturbation unit are in data communication with the homomorphic aggregator via the communication channel. The field unit includes a fluid channel and a sensor configured to sense a concentration of one or more elements of fluids flowing through the fluid channel. The concentration of the one or more elements of the fluid flow is sensed at the sensor, which generates a fluid data signal representative of the concentration of the one or more elements of the fluid flow, which is sent to the homomorphic aggregator via the communication channel. A perturbed data signal representative of a verification value is generated at the field site, and the perturbed data signal is sent to the homomorphic aggregator via the communication channel. The fluid data signal and the perturbed data signal are recorded at homomorphic aggregator to a blockchain.

In some embodiments, the method also includes the step of collecting a fee associated with a perturbation request.

### BRIEF DESCRIPTION OF THE SEVERAL VIEWS OF THE INVENTION

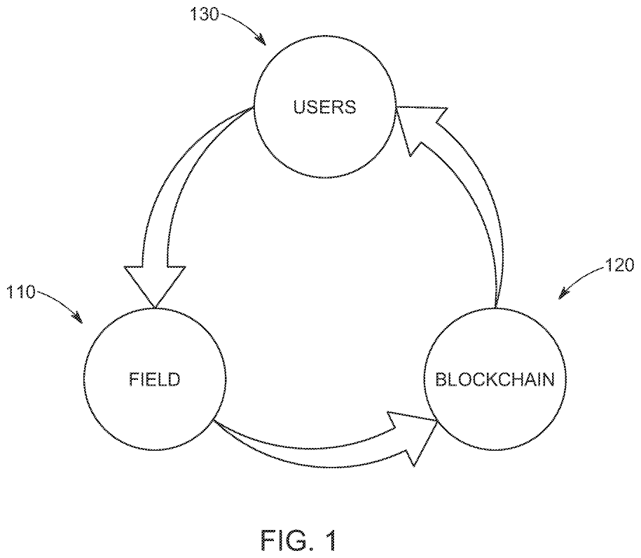

FIG. 1 is a schematic block diagram showing the relationship of the three classes which operate in the flow of data in the network;

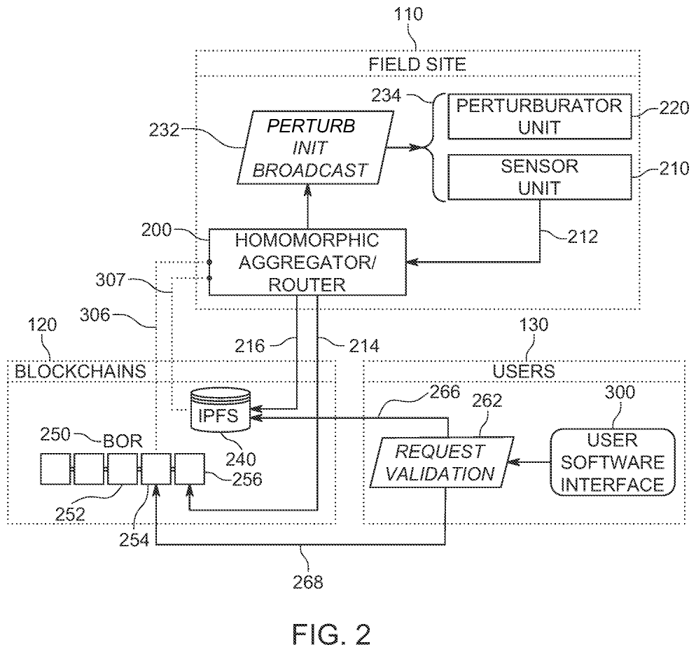

FIG. 2 is a schematic block diagram of an overview of an exemplary system that may be used by the invention, showing a networked data flow through all transformations from start to finish;

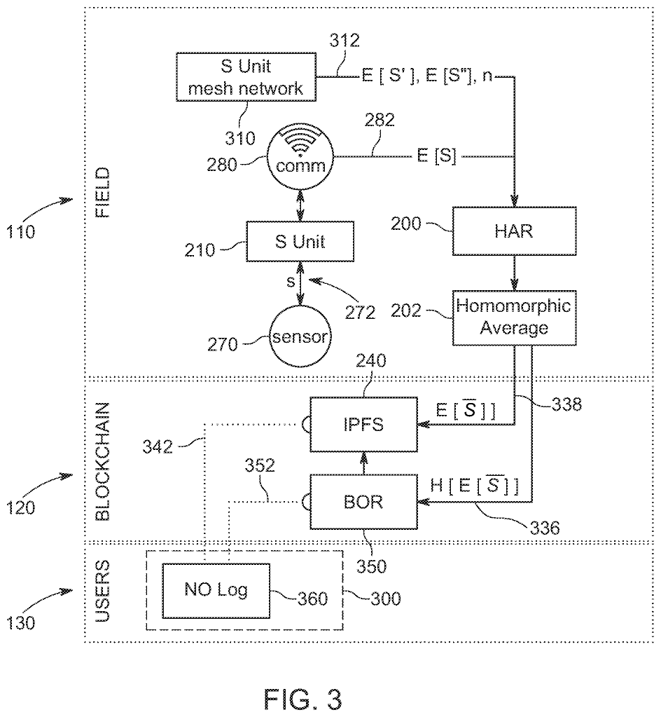

FIG. 3 is a schematic block diagram showing an overview of the normal mode operation in one embodiment of the invention, that may be used to collect regular readings of greenhouse gas emissions at a remote site and record date to a blockchain;

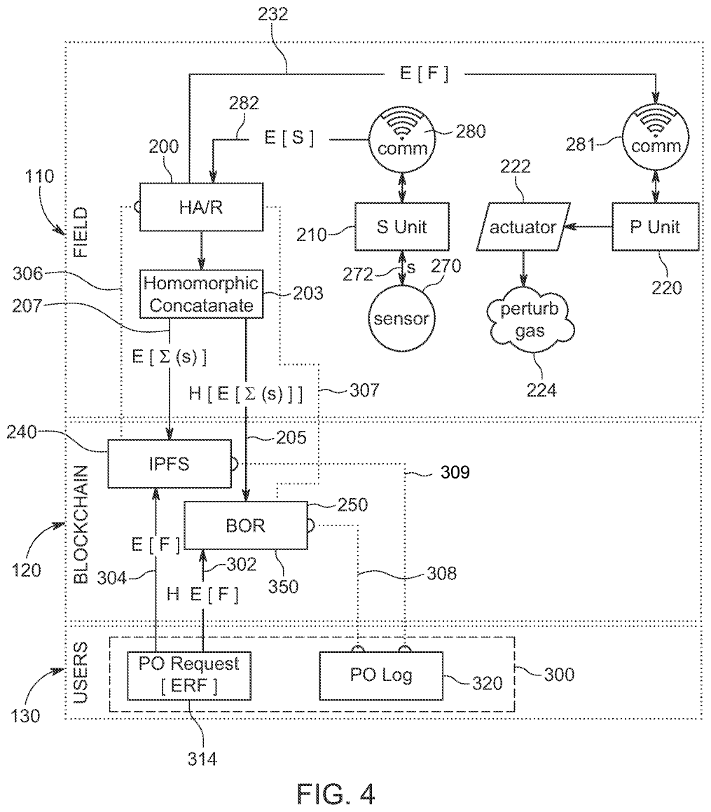

FIG. 4 is a schematic block diagram showing an overview of the perturbation mode operation in one embodiment of the invention, that may be used to validate trends of sensor readings of greenhouse gas emissions at a remote site;

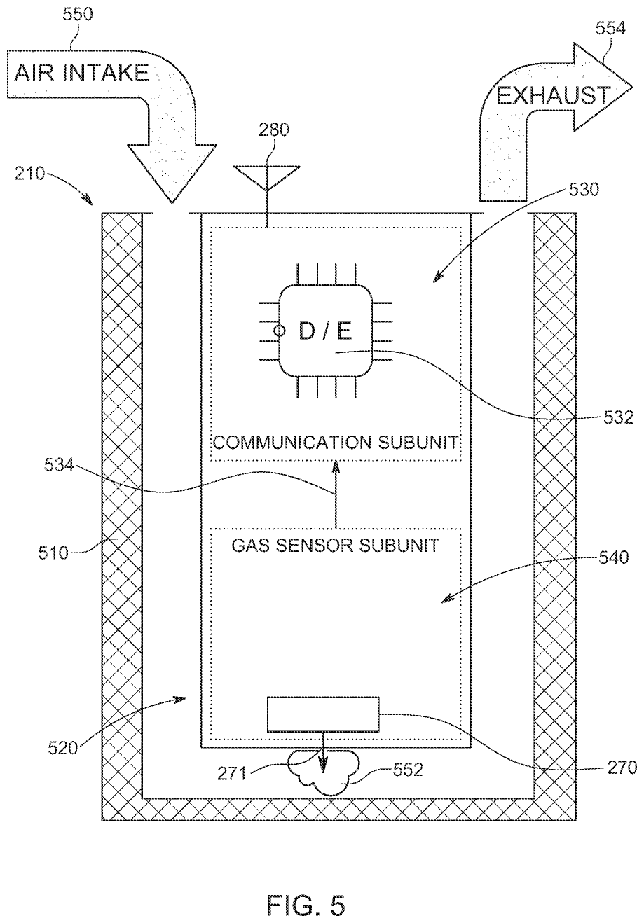

FIG. 5 is a schematic block diagram showing an overview of the primary functional components of the field installed sensor unit (S-unit);

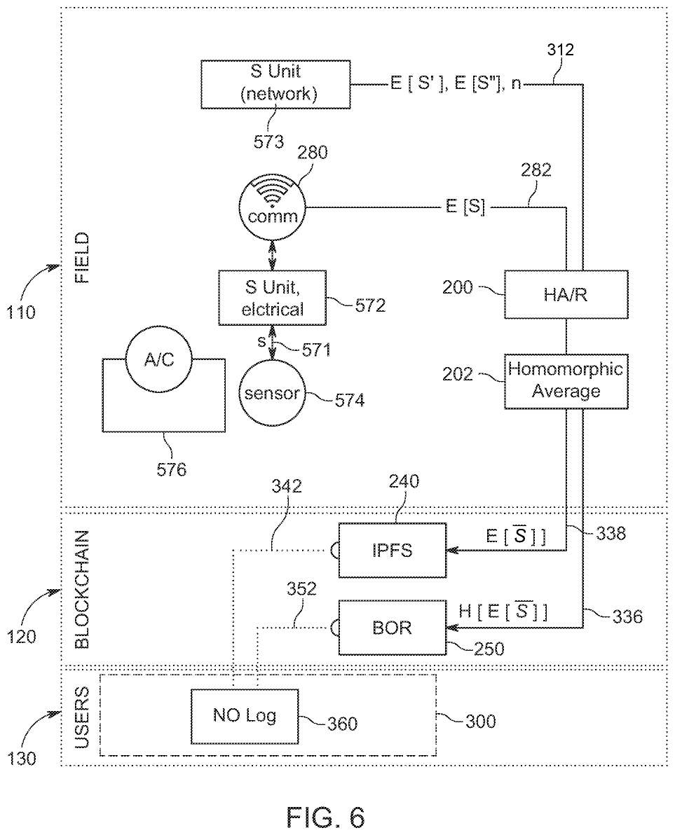

FIG. 6 is a schematic block diagram showing an overview of the normal mode operation in another embodiment of the invention, that may be used to monitor consumption of electricity at a field site and record date to a blockchain; and

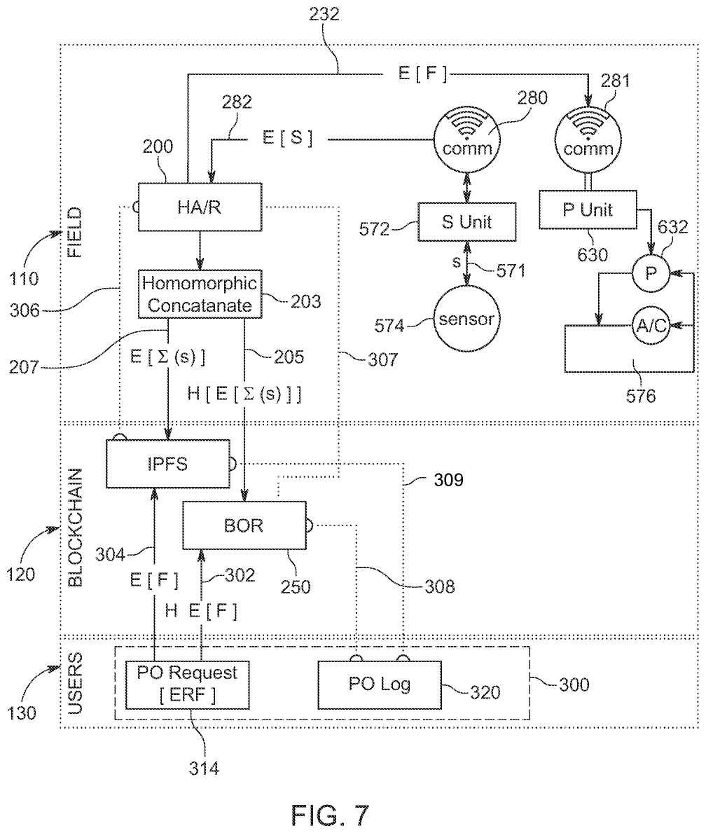

FIG. 7 is a schematic block diagram showing an overview of the perturbation mode operation in another embodiment of the invention, that may be used to monitor consumption of electricity at a field site and record date to a blockchain.

### DETAILED DESCRIPTION OF THE INVENTION

As shown in FIG. 1, three component classes are utilized for the MPM cycle to initiate and complete a data transport loop. These are users 130, field 110, and blockchain 120. Users 130 include entities such as producers/emitters, regulators, markets, and validators, where validators are human users who intercede in MPM cycles. Field 110 is a network of physical site-installed monitoring units and associated components, including sensors, dedicated encryption hardware, network hardware, and perturbation actuators. Blockchains 120 are the trustless record keepers of verified cryptographic data, which may include public proof of work ledgers such as Bitcoin, proof of stake ledgers such as Ethereum, and distributed file sharing ledgers, such as IPFS, or may involve similar distributed ledgers, whether currently existing or developed in the future.

FIG. 2 is an overview of the data transport network. It may make use of established cryptographic and infosec communication processes and utilize existing network infrastructure protocols as generally known in the art. The collaboration of these systems as described herein combine to create the measure-perturb-measure (MPM) process.

Field site components include sensor units 210, perturbation units 220, and one or more homomorphic aggregator routers (HA/R) 200. Components in the field site 110 communicate over a secured local area network (LAN), which may be a wireless ethernet, wired LAN, or wireless mesh network protocol. Sensor units 210 operate autonomously, broadcasting cryptographically formatted data packages as messages 212 to be gathered by an HA/R 200. HA/R 200 makes operation dependent summaries of all collected data, writing an archived journal of raw data 216 to a selected file distribution blockchain protocol, such as IPFS 240. Each HA/R 200 is provided with a public/private cryptographic key pair. The HA/R also hashes each journal and writes the hash output as a fingerprint 214 in a transaction to the next available block (shown as block 256) on the blockchain of record (BOR) 250. Blocks (for example, blocks 252, 254, 256) are written sequentially to the BOR 250. Any hashing operation may be used to create hash fingerprint 214. In preferred embodiments, an unkeyed cryptographic hash function such as MD5, SHA-1, SHA-2, SHA-256 is used to create hash fingerprint 214, although other generally-known hash types and hash algorithms may be used.

Users interact with the system via a software interface 300. The diagram shows the effects of a user issuing a request validation command 262. As shown, the validation request 262 comprises two signed transaction messages 266, 268 that are signed with the public key of a particular HA/R 200 of a field site 110. Message 266 is an encrypted command that is recorded on IPFS 240. Message 268 is a fingerprint hash of the request 262, to be recorded to the next available block (shown as block 254) on the BOR 250.

In the field site 110, the HA/R 200 polls the blockchains 240, 250 via poll requests 307, 306 respectively. Poll requests 307, 306 monitor IPFS 240 and BOR 250 for request validation transactions 266, 268. When a HA/R 200 discovers a message 266, 268 containing a validation transaction 262 encrypted with its public key, the HA/R decrypts the message using the HA/R's corresponding private key. HA/R 200 then selects one or more perturbation units (P-units) 220 installed in the local area, and formats an encrypted broadcast message 232 using the private key of the selected P-units 220 with the appropriate commands from perturbation request 314 encoded to those selected P-units.

The MPM Cycle, when combined with blockchain technology provides testability, which can be used to provide a combination of transparency and/or markets for financial instruments derived from that data.

### FIELD DESCRIPTION

The field site 110 is an abstraction for all site locations where measurements are collected, and a field site, or simply site, is a predefined area location where an emitter or producer agrees to cooperate with MPM monitoring. A field site 110 may be configured for wide area network (WAN) communication access to tie it into the LAN. The field site 110 may include proprietary hardware with control software and qualified installation.

The three system component types present at each field site are at least one Homomorphic Aggregator Router (HA/R) 200, at least one sensor unit (S-unit) 210, and at least one perturbation unit (P-unit) 220.

### Homomorphic Aggregator Router (HA/R)

For coordination and WAN communication, one or more dedicated site controllers with routers are typically employed. This dedicated system includes hardware and software to perform a specialized cryptographic function called homomorphic aggregation. Homomorphic encryption is a method of encryption that allows certain operations on ciphertext without the use of the decryption key(s). A homomorphism is a binary operator ⊕ satisfying E[x]⊕ E[y]=E[x+y] for some +, such as summation or concatenation. A homomorphic transformation will be decipherable by the key-holder. Homomorphic aggregation is a mathematical process that works with encrypted data inputs without requiring private keys. Homomorphic aggregation is described, for example, in Paillier, Pascal “Public-Key Cryptosystems Based on Composite Degree Residuosity Classes”. Advances in Cryptology—EUROCRYPT '99. Lecture Notes in Computer Science. Springer. 1592: 223-238 (1999), which is hereby incorporated by reference in its entirety. The invention makes use of this process to summarize the encrypted data packets 212 sent from the S-unit 210.

The HA/R 200 is a standalone functional system unit with a processor, encryption communication layer, and a router for local area network (LAN). A field site 110 makes use of one or more HA/Rs 200 to coordinate and collate data traffic from LAN to WAN.

The system generally includes at least one full node for the blockchain of record (BOR). The BOR 250 is a public blockchain where hashes of critical messages are stored for permanent record keeping and certain cross-functional needs. A node is server that keeps local copy of a distributed public ledger, similar to an Ethereum or Bitcoin blockchain. The node operates with dedicated software and can execute various blockchain functions including sending transaction to, and validating transaction from, the network staging-pools and the blockchain itself.

The HA/R 200 collects, and routes data produced at a field site 110. The HA/R 200 is typically connected to the internet by WAN to maintain connection to both the network users and public blockchains, although other forms of connection to the internet may be employed. The HA/R 200 may also utilize a local area network (LAN) to broadcast system messages to the wireless S-units 210 and P-units 220 which are usually invisible to the open web.

A Homomorphic Aggregator generally includes specialized software designed to execute cryptographic utilities. A Homomorphic Aggregator performs limited mathematical operations on encrypted cypher text without access to the private cypher key, as described above. In the MPM cycle, the HA/R 200 takes the measurement data from the distributed local sensor network 310, provided by S-units 210, and either averages their readings over a predetermined time interval, or concatenates the data into a digest, according to functional requirements determined by the network. The HA/R 200 may apply different aggregation functions to data during different operational modes, such as a time average during normal operation and concatenation during perturbed operation.

In one embodiment, the HA/R 200 is a computer system running an operating system such as Linux, with an Ethernet network interface. In other embodiments, HA/R 200 may be implemented on small chip-based computer systems such as a Raspberry Pi. For technical simplicity and further security, the HA/R unit 200 might include a wireless router, but could operate by conventional standalone, but dedicated, external router(s). Whether the router is physically assembled with the homomorphic aggregator in a single system, or it is standalone, the HA/R 200 is referred to as one abstraction in the role of the MPM cycle.

Every field site 110 includes at least one HA/R 200, but may have more than one, as network engineering requires. In preferred embodiments, HA/Rs 200 further include industry standard tamper-proof physical security elements to minimize potential for physical modification or sabotage of the HA/R.

### S-Unit

Referring to FIG. 5, a diagram of a sensor unit (S-unit) 210 is shown. The S-unit 210 is includes a secure enclosure 510 containing an ambient air channel 520, a communication subunit 530, and a gas sensor subunit 540.

S-unit 210 includes an air intake 550 in gas communication with ambient air channel 520, and an air exhaust 554 in gas communication with ambient air channel 520. The gas sensor subunit 540 contains a sensor 270 that measures the concentrations of one or more target gasses contained in the ambient air 552 flowing through ambient air channel 520, shown as measurement 271. The sensor 270 subunit relays sensor data via intra-unit secure message 534 to the communication subunit 530. The communication subunit encrypts incoming data 534 to an internal private key at encryption layer 532, which may be implemented in hardware or software. The communication unit may employ conventional network interface hardware 280 to broadcast encrypted data packets 212 to the LAN of field site 110.

S-units 210 are typically configured to relay individual data to one or more HA/Rs 200, according to an appropriate IoT protocol standard. The S-unit 210 includes a sealed electronics enclosure with an extended housing designed with an open channel 520 to promote unobstructed flow of ambient air, termed an air sampling loop. The sealed enclosure includes two specialized electronic subsystems: communication and sensing. In some embodiments, a gas flow other than ambient air may be sampled by an S-unit 210. In still other embodiments, open channel 520 may be a liquid sampling loop.

The air sampling loop 520 may benefit from an included fan or other air-handling features, designed to facilitate and stabilized the flow of air or gas through the S-unit 210.

In a preferred embodiment, communication subunit 530 includes a common low power transceiver solution 280, which is used to communicate with one or more dedicated HA/Rs 200. The communication subunit 530 preferably includes an encryption layer 532 to provide for the need of privacy and security for data transport throughout the MPM cycle. The sensing subunit 270 further includes a dedicated low-power sensor suitable for detection of a desired target gas or fluid 552, for example carbon dioxide (CO₂) or methane (CH₄). The subunit thereby provides for redundant tamper-protection to ensure data signals 534 between sensor 270 and communication subunit 530 are not intercepted or modified.

### P-Unit

As shown in FIG. 4, the MPM perturbator unit 220, or P-unit, perturbs gas concentrations or other parameters measured by a sensor 270 of an S-unit 210. A perturbation is a temporary and intentional alteration or modification to a default state to introduce a measurable reaction. In this application, perturbation influences a discreet measurement parameter or set of parameters, and observing measurements of perturbations permits determination of the quality of non-perturbed measurements.

P-unit 220 typically includes a sealed electronics enclosure and a mechanical gas trigger subunit. A communication subunit 281 of P-unit 220 provides communication between the P-unit and one or more HA/Rs 200. The communication subunit 281 may employ wired ethernet, or wireless Wi-Fi, Bluetooth, or similar radio network protocol to communicate with the HA/Rs 200. The communication subunit 281 includes an encryption layer capable of decrypting private mode switch instructions originating from users of user class 130 and communicated via encrypted requests 314.

Generally, a P-unit 220 is positioned near one or more S-unit 210 air intakes 550. The P-unit 220 controls the introduction of a controlled reference gas 224, which alters localized air readings proximate to an S-unit 210 in a predictable manner. In some embodiments, the P-unit 220 is comprised of a mechanical actuator 222 which triggers either the release of a stored gas, or the reaction or combustion of reagents to produce a gas 224 on demand proximate to air intake 550 of S-unit 210.

Both the S-unit 210 and the P-unit 220 may employ standard power supply solutions such as any combination of batteries, solar chargers, wired AC or DC power, or other power sources. In some embodiments, more than one type of power may be provided for system redundancy in the event of a power disruption. The units are designed to prevent tampering that can compromise system integrity intentionally or accidentally.

### Initializing and Security

Both the device enclosures of S-units 210 and P-units 220 and the internal electrical engineering may employ off-the-shelf or custom configured security to match or exceed industry standard protocols. It is important to the trustless operability of the system that S-units 210 and P-units 220 confidently resist tampering with the internal circuit between the sensor/actuator and the encryption chip. Communication integrity between subsystems, such as the encrypted communication subunit 280 and the gas sensor subunit 270, should be reliable and continuous. Units 210, 220 are preferably tamper-resistant or tamper-evident regarding physical sensor obstruction, airflow obstruction, and internal circuit security. Tampering with the S-units 210 or P-units 220 by physical/air-flow obstruction, or disruption of encrypted data, is protected against by the design of the MPM-cycle, so long as perturbations are sufficiently frequent and unpredictable, and maintenance issues regularly scheduled, infrequent, or idiosyncratic enough to be reliably distinguished from tampering by human or machine observers.

The invention includes the use of multiple initialization steps that should be done by a trusted party to preserve system integrity and prevent any untrusted devices from entering the network. The initialization steps place cryptographic material on the devices, initialize the devices, and secure the physical layer of the devices. Each unit 210, 220 has an embedded encryption layer. The encryption layer may utilize public-key cryptography with a public/private keypair, such as Elliptical-Curve Diffie-Hellman (ECDH). The private keys of S-units 210 and P-units 220 are preferably unknown to all users, including the validators and installation technicians.

When initialized, each unit 210, 220 signs a single-use initialization broadcast message, to be published to the BOR 250. After registering to the network, the unit will auto-initialize a perturbation cycle, and send encrypted messages 212 to its assigned HA/R 200 to record measurements to BOR 250.

### Normal Operation Mode

Once a field unit has been initialized and registered with a local site network, it becomes a member of a group of field units, called a field site 110.

Referring to FIGS. 3 and 5, an overview of a normal operation mode is shown. One or more S-units 210 take sensor reading S 272 at sensor 270 and broadcasts an internally formatted cryptographic data package 282 via its dedicated communication channel 280, which may be Wi-Fi, Bluetooth, or other radio network protocol, or on wired ethernet, as appropriate to the installation. A field unit comprised of multiple sensor units may be placed in a network 310, thereby providing a data stream 312 including data from a first S-unit, second S-unit, etc.

HA/R 200 gathers S-unit messages 280 and/or 312 from the network of S-units 310 and computes a homomorphic average, the output of which is written as two transactions 338, 336: a raw data journal 338 is written to IPFS 240, and a hashed fingerprint of that raw data 336 is written to BOR 250. In typical embodiments, user software 300 periodically polls the blockchains 240, 250 via poll requests 342, 352 respectively, to update the user's dashboard with data (e.g., 336, 338) regarding the normal operation (NO) log 360, which is made visible to the user. Additionally, user software 300 periodically polls the blockchains 240, 250 via poll requests 309, 308 respectively, to update the user's dashboard with data (e.g., 207, 205) regarding the perturbed operation (PO) log 320, which is also made visible to the user.

During normal operation, each S-unit 210 functions autonomously as determined by local clock pulse which is triggered internally by a unit's system clock. The internal sensor 270 takes a sample reading 271 of ambient air 552 passing through open air channel 520, or air loop, assisted by internal fan(s), if necessary. The sensor unit 270 of S-Unit 210 gauges a gas concentration, for example a CO₂ concentration, then signals the encrypted communication subunit 530 with the concentration reading via secure message 534. The communication subunit 530 adds necessary device or location identification information, timestamps, or metadata, and then encrypts a data package 282. The data package 282 is then broadcast as ciphertext via communication interface 280 to be received by network HA/R 200.

A HA/R 200 receives incoming encrypted messages 282, 312 from one or more S-units 210, 310 as assigned by field site configuration. The HA/R 200 performs a cryptographic average operation 202, resulting in an encrypted aggregate data packet 338 having the form

HA/R:E[S]ⁿ→E[S̅]

without ever exposing the source data S from encrypted data packets 282, 312. In preferred embodiments, the HA/R 200 writes this package 338 to a public blockchain 240 using the IPFS protocol, or to a similar distributed filesystem, and appropriate software. The InterPlanetary File System (IPFS) is a protocol and peer-to-peer network for storing and sharing data in a distributed file system. IPFS uses content-addressing to uniquely identify each file in a global namespace connecting all computing devices. A hash of the content stored serves as a Universal Resource Indicator (URI) or global address for that content.

The HA/R 200 also makes a hash digest 336 of the package, H [E [S]] and queues the hash as a transaction to a BOR 250 mempool to be recorded. Generally, a network running blockchain ledgers collect unverified transaction messages into a staging pool called a mempool. As the block validators print new blocks, transactions are selected to be captured in the new block and removed from the mempool.

The aggregate data package 338 written to IPFS is encrypted with a form of weakened encryption, or a “release” blockchain encryption, that enables incentivized miners to decrypt the data in a pre-determined average timespan. The time-release function and how it integrates into the MPM cycle is described below.

### Perturbation Operation Mode

The normal operation described above takes regular ambient gas readings on a schedule, and recording secure logs of the readings, to record data and reveal trends at a given site. Typically, verification of the data collected is desired by system users to ensure that the readings provided are accurate, as sensors could be tampered with or accidently obstructed, or could distribute erroneous data due to malfunction.

Referring to FIGS. 4 and 5, an overview of the perturbation operation (PO) cycle is shown. A user requests a validation request 262, shown as a perturbation validation request 314 via the user software console 300. The perturbation request 314 is recorded as two transactions 304, 302: encrypted command 304 is written to IPFS 240, and a hash 302 of that command 304 is written to the BOR 250. The HA/R 200 polls the blockchains 240, 250 via poll requests 306, 307, thereby monitoring blockchains 240, 250 for transaction records 304, 302. Encrypted perturbation requests 314 discovered on the blockchains by the HA/R 200 are passed via local area network as messages 232 to each P-unit 220, via communication subunit 281. HA/R 200 may employ an encrypted broadcast message 234 to both P-units 220 and S-units 210 at a field site 100. A P-unit with a private key matching the encrypted request 314 will be able to decrypt the command instructions; however, other P-units 220 and S-units 210 will not be able to decrypt request 314 due to lack of the correct P-unit private key, and thus will not receive the command instructions.

Upon receipt and successful decryption of command instructions, the P-unit 220 implements the requested perturbation of the ambient air by releasing a gas 224 during a perturbation interval. The HA/R 200 collects encrypted data packages 282 from registered S-units 210 as in normal operation, but for the duration of the perturbation interval, the homomorphic aggregation function preferably switches from homomorphic addition 202 (FIG. 3) to homomorphic concatenation 203 (FIG. 4).

The perturbation data cycle completes by writing a transaction of encrypted data journal 207 to IPFS 240 and a hash 205 of journal 207 to BOR 250. Finally, the user software 300 polls the blockchains 240, 250 via poll requests 342, 352 for signatures matching the originating private key, and collects the data to update the requesting user's local dashboard of software interface 300.

A perturbation may be made by releasing a reference gas 224 to change the concentration of the monitored gas 552 in the ambient air channel 520 of an S-unit 210. One embodiment of the invention may use CO₂ concentrations several factors higher than the normal gas readings, for example, at 1200 ppm, a density which is nearly three times higher than the current global mean CO₂ level. In other embodiments, a greater or lesser multiplier of CO₂ relative to the global mean CO₂ level may be employed.

In another embodiment, a non-monitored gas may be released into the air channel 520 to dilute the monitored gas 552. In a preferred embodiment of air sample flow dilution, an inert gas such as N₂ may be used. When thus perturbed, detected concentrations of the monitored gas 552 are reduced by a predicted amount, such as 25%, 50%, or 75% or more.

In another embodiment, a tracer gas may be produced which interferes with the measurement of the measured gas 552, for example a water vapor which absorbs light at some of the same wavelengths as CO₂ such that detected concentrations of the monitored gas 552 are reduced by a predicted amount, such as 25%, 50%, or 75% or more.

In another embodiment, a selectable input signal may be generated and provided to simulate a physical sensor input. For example, a resistor network may be provided to simulate a physical voltage provided by a CO₂ sensor and presented to the system in lieu of a CO₂ sensor voltage. In a preferred embodiment, such a simulated voltage is randomly selected from a range of values consistent with the emulated CO₂ sensor.

By comparing the sensor data 360 collected from normal mode operation to perturbation mode operation data 320, a third-party validators may be able to thereby verify that a particular field site 110 is accurately reporting sensor data, or identify a field site 110 where anomalies are present.

To prevent intentional tampering, perturbation mode operation as described above cannot be anticipated by any viewer other than the validating user making the request 314. The user may specify a time for the perturbation request to be implemented at a P-unit 220. Perturbation events are scheduled and paid for by a validator in real-time by creating an encrypted request function E[F] 304.

E[F] 304 is assembled using the validator software 300, by a validator user. A validator user may be a person. In some embodiments, a validator may be an external computer that is monitoring P-unit data. The validator selects one or more field sites 110 to test with a perturbation. The software controller selects (possibly at random) from the list of registered P-units 220 installed at the selected field site(s) 110. The software 300 builds a request in a dictionary listing each P-unit 220 from the selected site(s) 110 by public key, then encrypts a mode message 304 with the public key, decipherable only by the holder of the private key. P-units 220 are the sole holders of their private key of a private/public keypair, and therefore are the only systems that can decipher an encrypted message 262 containing an encrypted perturbation request 314 to that P-unit 220. The P-unit(s) 220 selected for perturbation are matched in the dictionary with an affirmative message requesting a perturbation operation, and that entry is encrypted to the public key of the corresponding P-unit 220 at field site 110. The remainder could be matched with a negative message, or simply not encrypted to.

The validator may also choose a time-lag, for how far in the future the date requested is to be encrypted in the final posted record. The duration of the time-lag after implementation of the perturbation request as scheduled by the user may effect the payment cost to the validator. Duration of the time lag is enforced statistically, by the difficulty of the public key chosen relative to computational capability at the time of the scheduled perturbation operation. This feature thereby enables market interactions by making data public after the intended time interval.

In one embodiment, the validator software then calls a smart contract running on the BOR, with a signed hash of the unencrypted function H[F] which will serve as the URI pointing to IPFS, where F will be stored eventually, to create a permanent record of the event. The contract may include a payment token, and a deposit, cost determined by the validator's chosen options and market rates. The transaction containing the H[F] is batched to the BOR transaction mempool, where the software monitors to ensure the transaction is accepted and written to the chain. The deposit is released back to the validator when (1) the hash-commitment H[F] is stored in the contract, (2) the result of the perturbation has been received, and (3) a proof is submitted to the smart contract that F the unencrypted function is present on IPFS.

Every HA/R 200 is a node for the BOR 250 and IPFS 240. If a new block added to the blockchain contains an encrypted E[F], the applicable HA/Rs 200 read the encrypted request record 304, 302 from the blockchains 240, 250 by polling requests 307, 306. Upon decryption of the encrypted request records 304, 302, the HA/R 220 will switch local homomorphic aggregation mode from normal (e.g. summation/averaging) as shown in FIG. 3 to perturb (concatenation) as shown in FIG. 4, and broadcast the E[F] to its dedicated LAN thereby relaying the message to each of the P-units 220 in association with the site 110 being perturbed.

All P-units 220 on the dedicated LAN receive the E[F]. If a message to a particular P-unit is present, that P-unit finds the message addressed to its public key and decrypts the ciphertext using the P-unit's private key. The clear text contains a message to either engage perturbation mode or do nothing.

In a preferred embodiment of the invention, all P-units that receive a message to perturb will trigger an actuator to cause an increase in CO₂, releasing it to mix with the air that will be taken into nearby S-unit sensor air channel.

During perturbation mode, the HA/R 200 receives gas readings from its dedicated S-units 210 as per normal, but instead of averaging the measurements, the HA/R performs a data concatenation 203. After a suitable duration of time for the perturbation gas to diffuse, the HA/R will switch back to the normal operation mode shown in FIG. 3.

Referring to FIGS. 6 and 7, another embodiment including an electronic measurement and perturbation is shown, wherein the MPM cycle is configured to monitor the consumption of electricity at a field site 110. To configure a field site to monitor electrical use over time requires at least one electrical P-unit 630 and one or more electrical S-units 572. P-units 630 and S-units 572 are in data communication with HA/R 200 as described above.

FIG. 6 depicts a network data cycle similar to FIG. 4, and wherein electrical S-unit 572 is configured to read electrical power consumption instead of gas concentration levels. An electrical sensor 574 is placed inside of an electric power circuit 576. A power sensing S-unit 572 includes a sensory subunit to support an electrical power monitoring sensor 574. Electrical power monitoring sensors are well understood, often clamping to the neutral return line anywhere on a circuit to place the sensor in parallel with the load or loads being monitored. A field unit comprised of these sensor units may be placed in a network 573, thereby providing a data stream 312 including data from a first S-unit, second S-unit, etc. Data 312 concerning electrical usage is collected by HA/R 200 during normal operation and recorded to a blockchain 240, 250.

FIG. 7 depicts the perturbation cycle of an electrical MPM data cycle, which differs from FIG. 5 gas MPM by utilizing electrical S-units 572 and by having an electrical perturbation unit 630.

A perturbation in an electrical power monitoring field embodiment is managed by a P-unit 630 with the same functional role as the gas embodiment P-unit 220. P-unit 630 responds to a perturbation request 314 by perturbing the electrical circuit 576 by introducing a perturbation electrical load 632. The electrical P-unit 630 utilizes the same general communications and cryptographic hardware and routines that the gas/fluid P-unit 220 utilizes.

The perturbation the electrical P-unit 630 provides is to provide a change in the electrical load, for example adding a controlled resistive load 632 in parallel to the circuit being tested. An additional resistance of known quantity applied to an active electrical circuit will cause predicable changes in relationship to the existing load, the user load, in demand on the circuit.

The data 282, 312 being secured to a blockchain record is the amount of power consumption on an electrical network. The perturbation provided by resistive load 632 to electrical load 576 in response to an encrypted perturbation request 314 may be, for example, a combination of resistors selected in powers of 2 (e.g., R₁ having a resistance of 1 ohm, R₂=2 ohms, R₃=4 ohms, R₄=8 ohms, etc.) By selecting a combination of the individual resistances R₁, R₂, R₃, R₄, etc., and placing the selected resistors in a series configuration, the resistive load 632 is representative of a binary number of sufficient size to be difficult to predict, and the resulting perturbed measurement is the live reading from an ammeter or voltmeter coupled to electrical load 576. In preferred embodiments having n resistors R₁ . . . Rₙ, n is at least 8, and more preferably at least 16, and still more preferably at least 32.

In another embodiment suitable for higher-power applications in which it is not safe to introduce resistive losses, the P-unit 630 is situated on a separate circuit, and the S-unit 572 is a secured solenoid through which both the P-unit circuit and the measured-circuit's power line passes. In this way, perturbations will show up in recorded data 282, without disrupting the operation of the measured circuit. The S-unit's 572 topological relationship with the P-unit circuit 630 and the measured circuit 576 is preferably physically secured and/or tamper-evident.

In some embodiments, the data aggregated by the HA/R 200 may include the power usage of multiple different device types, which may be distributed over multiple sites, for example individual residences or industrial sites. The aggregation function 202 may sum power usage of similar device types over distinct sites, while not collecting data regarding individual devices. In such embodiments, the data which can be unencrypted from the BOR 250 is the sum of each device type over the separate sites.

The field control of an electrical power MPM cycle may include the option for a local agent to trigger a perturbation cycle. Local control may be provided, for example, by a dedicated terminal accessed at the HA/R. With the proper authentication, a local agent may request one or more P-units 630 in the field site 110 to initiate a perturbation cycle and yield reports directly and/or through the extended data transport chain, i.e., to the blockchain of record 250.

In some embodiments a time-delayed automated perturb-cycle, triggered by a change in state, may be used. In such an embodiment, a request to switch from a High-Power to Low-Power mode is registered, such as a request from a power company to an electrical consumer, and a random future time (for example, an offset by a number of seconds) within a time period is determined. For example, the time-period may be within the next hour, and may be determined by the hash of the microsecond timestamp at which that request is made, modulo 60*60. A perturbation cycle is then scheduled at that time. Such embodiments thereby allow an electrical utility to test, for example, whether an electrical customer's energy usage has been switched from a normal power mode into (and/or remained in) low-power mode in response to a request by the utility to conserve or limit power consumption.

### Data Transport Loop

The MPM cycle relies on discrete messages between the system's coordinated components. Messages may be control signals, software/firmware updates, identity verification and registration signals, or data payload packages. Data payload in the MPM cycle may include the gas measurement value, timestamp, id signatures, mode attribute, and other system attributes such as software version.

The MPM cycle validates and verifies the gas reading, a numeric value with associated identifiers. As this data must be made both private and secure, qualities that make the data valuable to the users, the entire network uses well-understood encryption technology for all messages. Pertinent data is sorted to specified blockchains for record keeping and post collection functionality.

### Messages

Each system class, Users, Field and Blockchains, may use the WAN to send output messages as input to the next class in the cycle. Users message the Field and Blockchain classes. The Field sends messages only to Blockchains, and the Blockchain provides essentially passive messages (records and receipts), which Users and Field controllers reference to initiate or terminate data transport cycles.

### Time-Lock Messages

Validators contracting for validated data gain exclusive access to the data for the period in which it remains encrypted. A preferred embodiment of the invention uses a market program to incentivize blockchain miners to work on deciphering intentionally low-difficulty encrypted data. Time-lock encryption may be used to render encrypted data-packets decipherable within a projected time interval. Suitable mechanisms for time-lock encryption and release of data contained therein are generally known in the art.

### Exemplary Use of Systems and Methods for CO₂ Monitoring

A sector of interest for users of the present invention is users needing to monitor emissions from large industry suppliers, such as power producers, resource extractors, and manufacturers. Users could include government regulators, third-party validators, secondary market traders, and the emitter producers (polluters) themselves. Gases such as carbon dioxide (CO₂) and methane (CH₄), so-called greenhouse gases, may be classified as pollutants, and as such may fall under local, federal and international regulatory control, and therefore may need to be monitored to provide proof of compliance by those polluters.

In one embodiment of the invention, a site-monitor system is deployed for tracking CO₂ emissions. The site monitoring system is composed of secure field-installed hardware/software packages configured to perform independently, relaying required data securely using a defined protocol, allowing for necessary coordination from system administrators and users.

The site-monitor system continuously measures the classified controlled gas under normal operations, and periodically perturb the reading by the release of a reference gas, that is, change the local gas reading in a predictable manner. Data collected throughout the process is secured, aggregated, and relayed through to a data transport process as described above.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description and all changes that come within the meaning and range of equivalency of the claims are therefore intended to be embraced therein.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A system for monitoring and reporting a fluid concentration, comprising:
a homomorphic aggregator including a processor;
a field unit including a processor, a fluid channel, and a sensor configured to sense a concentration of one or more elements of a fluid flow through the fluid channel, wherein the field unit is in data communication with the homomorphic aggregator via a communication channel; and
a perturbation unit in fluid communication with the fluid channel, wherein the perturbation unit comprises an actuator configured to alter the concentration of the one or more elements of the fluid flow through the fluid channel, and wherein the perturbation unit is in data communication with the homomorphic aggregator;
wherein the field unit is configured to communicate a fluid data signal to the homomorphic aggregator via the communication channel, wherein the fluid data signal is representative of the concentration of the one or more elements of the fluid flow;
wherein the perturbation unit is configured to receive a perturbation request and perturb the fluid flow to the field unit such that the field unit provides a perturbed data signal to the homomorphic aggregator representative of a perturbed fluid concentration;
wherein the homomorphic aggregator is configured to record the fluid data signal and the perturbed data signal to a blockchain record.

2. The system of claim 1, wherein the fluid flow is a gas.

3. The system of claim 1, wherein the field unit is configured to encrypt the fluid data signal prior to communicating the fluid data signal to the homomorphic aggregator.

4. The system of claim 1, wherein the perturbation request is encrypted using a private key of the perturbation unit.

5. The system of claim 1, wherein the homomorphic aggregator is configured to receive the perturbation request and communicate the perturbation request to the perturbation unit.

6. The system of claim 1, wherein the perturbation unit is configured to receive the perturbation request and communicate the perturbation request to the homomorphic aggregator.

7. The system of claim 1, wherein the homomorphic aggregator is configured to record the fluid data signal to a blockchain record at a first sampling rate and record the perturbed data signal to the blockchain record at a second sampling rate.

8. The system of claim 7, wherein the second sampling rate is higher than the first sampling rate.

9. The system of claim 1, wherein the fluid data signal is added to the blockchain record using a first aggregation function, and the perturbed data signal is added to the blockchain record using a second aggregation function.

10. The system of claim 1, where the blockchain record includes a perturbed data stamp.

11. The system of claim 1, wherein the one or more elements of the fluid flow includes carbon dioxide.

12. The system of claim 1, wherein the one or more elements of the fluid flow includes methane.

13. The system of claim 1, wherein the fluid flow is water, and the element of fluid flow being measured is a pollutant or other chemical of interest for water quality monitoring.

14. The system of claim 1, wherein the blockchain record includes a time stamp.

15. A system for trustlessly monitoring a signal, comprising:
an electrical source;
an electrical load coupled to the electrical source;
a sensor module including a load measurement sensor coupled to the electrical load and to a controller;
and a homomorphic aggregator in data communication with the controller and the sensor module;
wherein the sensor module is configured to transmit data representative of a first operating condition of the electrical load to the homomorphic aggregator, and wherein the homomorphic aggregator records the data representative of the first operating condition to a blockchain;
wherein the controller is configured to receive a perturbation request;
wherein the controller is configured to electronically modify the electrical load presented to electrical source in response to the perturbation request; and
wherein the sensor module is configured to transmit perturbation data representative of a second operating condition to the homomorphic aggregator, and wherein the homomorphic aggregator is configured to record the data representative of the second operating condition to the blockchain.

16. The system of claim 15, wherein the controller receives the perturbation request from the homomorphic aggregator.

17. The system of claim 15, wherein the controller receives the perturbation request from an external source.

18. The system of claim 15, wherein the homomorphic aggregator is configured to add the data representative of the first operating condition to the blockchain using a first aggregation function, and is configured to add the data representative of the second operating condition to the blockchain using a second aggregation function.

19. A method of monitoring and reporting a fluid concentration, comprising:
providing a homomorphic aggregator including a processor and an encrypted communication channel;
providing a field unit including a fluid channel and a sensor configured to sense a concentration of one or more elements of a fluid flow through the fluid channel, and wherein the field unit is in data communication with the homomorphic aggregator via the encrypted communication channel; and
providing a perturbation unit;
providing a fluid flow to the sensor;
sensing at the field unit the concentration of the one or more elements of the fluid flow and generating a fluid data signal representative of the concentration of the one or more elements of the fluid flow;
sending the fluid data signal to the homomorphic aggregator via the encrypted communication channel;
receiving at the perturbation unit a perturbation request;
generating at the field unit a perturbed data signal representative of a verification value;
sending the perturbed data signal to the homomorphic aggregator via the encrypted communication channel;
recording at homomorphic aggregator the fluid data signal and the perturbed data signal to a blockchain record.

20. The method of claim 19, further comprising the step of collecting a fee associated with the perturbation request.
